(19) United States
(12) Patent Application Publication
(10) Pub. No.: US 2021/0114024 A1
(43) Pub. Date: Apr. 22, 2021

(54) Device and Method for Preparing Sample Material

(71) Applicant: Robert Bosch GmbH, Stuttgart (DE)

(72) Inventors: Franz Laermer, Weil Der Stadt (DE); Jochen Hoffmann, Renningen (DE); Tino Frank, Luzern (CH); Paul Kallenberger, Leonberg (DE)

(21) Appl. No.: 17/041,868

(22) PCT Filed: Apr. 24, 2019
(86) PCT No.: PCT/EP2019/060435

§ 371 Date: Sep. 25, 2020

(30) Foreign Application Priority Data
Apr. 26, 2018 (DE) 10 2018 206 469.5

(51) Int. Cl. B01L 3/00; C12M 1/26

(52) U.S. Cl. CPC B01L 3/502715; C12M 33/04; B01L 2200/027; B01L 2400/0487; B01L 2300/044; B01L 2300/0672; B01L 2300/0681; B01L 2200/0689

(57) ABSTRACT

A device for preparing sample material is designed as a rotary device by means of which a defined quantity of liquid can be drawn in into a sample receiving chamber for the sample material or can be expelled from the sample receiving chamber for the sample material, by means of a rotary motion.

The invention relates to a device and a method for preparing sample material. The invention further relates to a microfluidic system having such a device.

### PRIOR ART

The German laid-open specification DE 10 2005 050 347 A1 discloses a sample removal device, in particular a biopsy needle, composed of a hollow needle, having a distal opening with a peripheral edge, and of a stylet which is guided displaceably in the hollow needle and has a tip and a length such that the tip can protrude from the distal opening of the hollow needle. The sample removal device is configured, for example, as a fine needle biopsy device. The sample removal device or fine needle biopsy device is used to remove animal, human and/or plant tissue. In cases of suspected disease, fine needle biopsies are performed to remove tissue material or cells from the lungs, thyroid gland or prostate, for example. This sample material is traditionally placed onto a slide and assessed by a pathologist. The assessment involves visual examination of the morphology of the cells, for example. Cell-specific features are identified by what is known as immunohistochemical staining. In addition, genetic features of the cells are also increasingly being determined. The sample preparation steps that are needed for this are in most cases extensive and, consequently, are often not carried out directly. In some cases, this leads to treatments being prescribed without knowledge of relevant mutation states.

### DISCLOSURE OF THE INVENTION

The device for preparing sample material is advantageously configured as a rotary device with which, by means of a rotation movement, a defined quantity of liquid can be drawn into a sample-receiving space for the sample material or can be expelled from the sample-receiving space for the sample material. The sample-receiving space preferably has a microfluidic volume, in particular a liquid volume, of one to one thousand microliters, preferably between ten and one hundred microliters. The sample material is, for example, animal, human and/or plant tissue. The sample material is removed, for example, with a suitable sample removal device. The sample removal device is, for example, a biopsy needle or a biodetector with a functional or functionalized surface. The functional or functionalized surface advantageously serves to isolate molecules or cells from the human body. Cells, in particular tumor cells, circulating in the blood stream can be removed from a patient with the biopsy needle. The functional or functionalized part of the biopsy needle is advantageously coated such that either cell-free DNA or cells of epithelial origin, expressing a defined surface protein such as EpCAM, come into contact with the needle surface and are bound by antibodies present there, such as anti-Ep-CAM. The abbreviation Ep-CAM stands for epithelial cell adhesion molecule. In one use, the biopsy needle or the biodetector is introduced for example for thirty minutes into the brachial vein of a patient, removed and washed. A physician then determines the number of fixed cells and/or determines the mutation state of the cells. In a microfluidic system, small sample quantities can be analyzed with a high degree of sensitivity. Automation, miniaturization and parallelization additionally permit a reduction in the number of manual steps and, consequently, fewer errors caused by such steps. Prior to a microfluidic analysis process, however, the macroscopic sample must first of all be transferred into the microscopic or fluidic fluid environment.

This so-called world-to-chip interface routinely demands the preparation of an input solution or input sample. Typically, the sample preparation is carried out off-chip—manually or with another device. As a result of the transport of fluid between different vessels and the use of different solvents and washing steps, this has the effect that the in most cases already concentrated sample is further diluted. There is also the danger of contamination or decomposition of the fragile sample material as a result of time-intensive steps.

A point-of-care analysis, which most lab-on-a-chip applications provide, demands rapid sample analysis without complicated and labor-intensive working steps, which are normally performed only by trained personnel in central laboratories.

Microfluidic systems are often needed for different uses. A universal network system permits the analysis of different problems. The difference between the various approaches often involves another type of sample preparation. To this end, the microfluidic analysis unit would often have to be newly adapted, which is associated with high development costs. If, for example, a sample from a needle biopsy is intended to be transferred into a system for a liquid sample, a solution has to be found for the pre-processing a rigid needle without the microfluidic analysis unit having to be modified at high cost (e.g. new injection molded parts). Otherwise, the advantage of the almost completely automatic processing is lost.

With the claimed rotary device, it is possible for small well-defined quantities of different liquids to be quickly, easily and precisely drawn up and then expelled again, in order thereby to transfer immobilized samples microfluidically into the suitable input form. Thus, an immobilized sample can be transferred into a suitable solution, suspension or dispersion. For this purpose, it has to be rinsed among other things, but the sample material also has to be detached or cell material released by lysis. This small sample volume can then be transferred directly into a microfluidic system for further processing.

The claimed rotary device is preferably connected in an airtight manner to a cannula-shaped volume. The sample is placed in this volume. By means of a predefined rotation, a defined quantity of liquid can be injected into the cannula volume and expelled. This system affords the following advantages: The device has an intuitive and straightforward user design. This also permits handling by personnel who have not been specially trained. In addition, it minimizes the danger of possible errors that could be made by the user. Volumes are predefined and cannot be altered and are defined purely by the complete rotation procedure. Furthermore, the use of transparent cannulas, for example made of glass, permits visual monitoring of the handling procedures, which provides additional handling safety. The use of transparent cannulas, for example made of glass, also advantageously permits an optical analysis of the sample material, for example a cell count with the aid of a microscope, wherein the sample material is located in a protected environment (in the interior of the cannula). Through the use of a cannula, the system is configured such that it is also possible to work without loss of material and in a manner free from dilution. Rigid forms, for example a swab, a wire, a needle or a fine punching tool for a biopsy, can be introduced into the cannula form without the geometry thereof having to be modified. Moreover, the geometry of the microfluidic system does not have to be specially adapted. This allows existing microfluidic systems to be supplemented with the sample preparation system described here, without adapting the system. The described system is substantially closed. In this way, the risk of contamination is reduced by several factors. The described system can be realized by assembly of a small number of cost-effective and disposable parts. This permits a once-only use, which is desirable and routine in the medical sector. Solutions for cleaning and for prevention of cross-contamination do not need to be developed. By drawing in and expelling the desired quantity of liquid, it is possible to reduce the likelihood of air bubbles appearing in the sample liquid. For a subsequent analysis, for example on a microfluidic platform, it is much easier to transfer a liquid sample onto the chip in a bubble-free manner than to convert a rigid sample on the microfluidic platform into liquid in a bubble-free manner. The chemicals required for the washing or for the lysis could, for example, already be stored on a chip. This enhances the degree of user friendliness, since the number of the individual parts of the overall system is minimized. The use of the described invention is based on concepts known to the target users. The operation of tools with similar functions, such as syringes or pipets, is entirely familiar to personnel working closely with patients (for example nurses, physicians, paramedics), and therefore no further training on the topic is needed. The system can be built up by combination of commercially available Luer parts that are used as standard in medical fluidics. These parts are present as standard and can be adapted by slight modifications to commercially available components (for example integration of seal, abutment location). If samples cannot be analyzed directly in situ, the device permits an initial simple sample preparation, which transfers the analyte in the small volume into a stable form (for example fixed DNA) and can then be dispatched in the small volume along with the adapter. The device can be further used in order to remove a small volume again from a microfluidic system and use it for further analyses. This is of interest if results are further evaluated in clinical studies. Thus, a specific result can be directly processed for sequencing.

A preferred illustrative embodiment of the device is characterized in that the sample-receiving space comprises a volume that is smaller than ten milliliters. For example, the sample-receiving space has a volume of approximately ten to two hundred microliters.

A further preferred illustrative embodiment of the device is characterized in that the rotary device comprises a rotary body which, by way of a thread, is coupled to a liquid-receiving space with a volume whose size is altered by rotation of the rotary body relative to the thread or by rotation of the thread relative to the rotary body. The volume of the liquid-receiving space is greater, preferably at least two to three times greater, than the volume of the sample-receiving space. The rotary body advantageously comprises, radially to the outside, an outer thread which complements an inner thread of the aforementioned thread. In addition, the rotary body is advantageously provided with a central through-hole which connects the liquid-receiving space fluidically to the sample-receiving space. According to a further illustrative embodiment, the rotary body is configured as a Luer lock element. Depending on the direction of rotation of the relative rotation movement, the volume of the liquid-receiving space becomes smaller or greater. If the volume of the liquid-receiving space becomes smaller, fluid, in particular liquid, is expelled from the liquid-receiving space through the sample-receiving space. If the volume of the liquid-receiving space becomes greater, liquid is drawn into the liquid-receiving space through the sample-receiving space. The sample material can thus be easily subjected to preparation steps such as washing, fixing and/or lysis. As soon as the preparation steps are completed, the sample now present in liquid form can be transferred easily, in particular reproducibly, by rotation of the rotary body. The liquid sample is expelled here from the sample-receiving space. Thus, the sample can be advantageously transferred directly to a lab-on-a-chip. Depending on the set-up, a liquid used for the washing, fixing or lysis can also already be stored in the lab-on-a-chip. With the claimed device, the required liquids in a corresponding microfluidic system can also be removed from the lab-on-a-chip. As regards this illustrative embodiment, the thread is advantageously stationary when the rotary body is rotated. If the thread is rotated, then the rotary body is advantageously stationary. Of course, the two parts, i.e. the rotary body and the thread, could also both be rotated relative to each other. In this context, a thread designates in particular an inner thread portion which meshes with an outer thread portion, which is in turn formed on the rotary body. The inner thread portion is formed, for example, in a hollow body in which the rotary body is rotatable.

A further preferred illustrative embodiment of the device is characterized in that the liquid-receiving space is delimited by a hollow body which is equipped internally with the thread. The hollow body has, for example, the shape of a straight circular cylinder, which is closed at one end. The rotary body is rotatable in the hollow body via the thread, such that the volume of the liquid-receiving space delimited by the hollow body changes when the rotary body is rotated in the hollow body. The closed end of the hollow body can be configured as a Luer lock element. A non-functional or non-functionalized portion of a biopsy needle can advantageously be guided out from the liquid-receiving space through the Luer lock element. The biopsy needle is advantageously arranged with its functional or functionalized portion in the sample-receiving space. Depending on the configuration of the biopsy needle, the biopsy needle can extend through the liquid-receiving space through the closed end of the hollow body which is advantageously configured as a Luer lock element. Of course, the hollow body can also be rotated relative to the rotary body in order to reduce or enlarge the volume of the liquid-receiving space in a defined manner. The rotary body can then be secured for example with one hand, while the hollow body is rotated with the other hand.

A further preferred illustrative embodiment of the device is characterized in that the hollow body has a sealed push-through region at an end directed away from the sample-receiving space. The hollow body has, for example, the shape of a straight circular cylinder, which is closed at one end. The rotary body is rotatable in the hollow body via the thread, such that the volume of the liquid-receiving space delimited by the hollow body changes when the rotary body is rotated in the hollow body. The closed end of the hollow body can be configured as a Luer lock element. A non-functional or non-functionalized portion of a biopsy needle can advantageously be guided out from the liquid-receiving space through the Luer lock element. The biopsy needle is advantageously arranged with its functional or functionalized portion in the sample-receiving space. Depending on the configuration of the biopsy needle, the biopsy needle can extend through the liquid-receiving space through the closed end of the hollow body which is advantageously configured as a Luer lock element.

A further preferred illustrative embodiment of the device is characterized in that a biopsy needle, as shown in FIG. 14, is inserted into a lysis device. However, the latter has no rotary device for receiving liquid, but instead a pipetting ball (or similar) mounted for example at location 153. By means of this ball, process liquids can be introduced into the capillary 15 or expelled by an increase or reduction in volume. This simplifies the fluid actuation unit by comparison with the aforementioned rotation mechanism.

A further preferred illustrative embodiment of the device is characterized in that the liquid-receiving space is fluidically connected to the sample-receiving space. It is thus simple to ensure that liquid can be drawn into the liquid-receiving space via the sample-receiving space. At the same time, liquid can be expelled from the liquid-receiving space through the sample-receiving space.

A further preferred illustrative embodiment of the device is characterized in that the sample-receiving space is delimited by a tube body which is open at its end directed away from the liquid-receiving space. Liquid can be drawn in or expelled through the open end of the tube body. The tube body is either way configured similarly to a cannula or capillary.

A further preferred illustrative embodiment of the device is characterized in that the tube body or the rotary body has an outer thread portion which is designed complementing a inner thread portion arranged in the liquid-receiving space. The tube body is advantageously combined with the rotary body. The tube body can be connected integrally to the rotary body. The outer thread portion is advantageously formed on a collar which is angled away from the tube body or a main body of the rotary body. The tube body can also have an open end with a tip which is either way configured similarly to a pipet tip. The rotary body, for example a cannula or a capillary, can then be fluidically connected to the rotary body with a suitable sealing device.

A further preferred illustrative embodiment of the device is characterized in that the tube body, the hollow body and/or the rotary body are/is combined with at least one sealing device The sample-receiving space and the liquid-receiving space, apart from the open end of the tube body, can thus be sealed off from the environment in a fluid-tight manner, in particular in an airtight manner.

A further preferred illustrative embodiment of the device is characterized in that the tube body, the hollow body and/or the rotary body are combined with a filter device. With the filter device, individual method steps such as purification or pre-purification of the sample material can be implemented more effectively.

A further preferred illustrative embodiment of the device is characterized in that the liquid-receiving space or the sample-receiving space has an attachment for the delivery and/or discharge of a fluid. The attachment is provided, for example, on an attachment body of the rotary device. However, the attachment can also be provided on a or the hollow body, which delimits the liquid-receiving space. However, the attachment can also be provided on an additional part, for example on a T-piece, as is further described below. The attachment advantageously serves to deliver a fluid in order to remove all the sample material from the liquid-receiving space and the sample-receiving space. A device is thus advantageously created for the loss-free, microfluidic preparation of immobilized sample material, in particular on rigid needles, preferably by means of a two-phase system. A fluid, in particular an oil phase, can be delivered via the attachment in order to remove a sample liquid completely from the sample-receiving space, for example from a cannula, and to transfer it, without loss, to a lab-on-a-chip platform. A sample liquid designates a liquid that contains sample material. In addition, the attachment can serve to perform partial tasks involved in the storage and provision of a liquid.

A further preferred illustrative embodiment of the device is characterized in that the attachment for the delivery and/or discharge of the fluid is provided as a third attachment on a T-piece, which delimits the liquid-receiving space and/or the sample-receiving space. The third attachment is advantageously closeable by a closure body. In the operation of the device, the closure body advantageously serves to tightly close the third attachment, particularly when no fluid is being delivered or discharged via the third attachment. The closure body is advantageously configured as a Luer lock element. The T-piece with the third attachment is advantageously likewise configured as a Luer lock element. The configuration as a Luer lock element or Luer lock elements simplifies the handling of the device.

A further preferred illustrative embodiment of the device is characterized in that the T-piece has a first attachment for the rotary device, a second attachment for the sample-receiving space, and the third attachment for the delivery and/or discharge of the fluid. In relation to the T-piece, the third attachment can be arranged transversely with respect to the first two attachments. However, in relation to the T-piece, the third attachment can also be arranged parallel to the first two attachments. Instead of a closure body, a syringe with a Luer attachment can also be screwed onto the third attachment of the T-piece or T-element. Fluid, in particular oil, can be easily applied via the syringe, by means of a piston of the syringe being actuated. The syringe can be mounted on the third attachment of the T-piece orthogonally but also parallel to a capillary or cannula.

The invention further relates to a microfluidic system having an above-described device for preparing sample material. In addition to the above-described device for preparing sample material, the microfluidic system additionally comprises at least one microfluidic chip. In this way, fully automated analysis of biological samples is easily permitted directly at the point of care. Reagents are advantageously present in the fluidic chip. With the reagents, the sample material can then be prepared, for example washed. The microfluidic chip advantageously comprises a sample input region. The sample input region on the microfluidic chip comprises an input channel, for example, which is suitable for receiving the open end of the tube body of the above-described device. For this purpose, the shape of the input channel is advantageously adapted to the shape of the tube body at its open end. The input channel in the microfluidic chip is connected to a microfluidic network at at least one connection site. This greatly facilitates the preparation or processing of the sample material, for example the washing and staining of the sample material.

The invention optionally also relates to a tube body, a hollow body, a rotary body, a sealing device and/or a filter device for an above-described device for preparing sample material or for an above-described microfluidic system. The stated parts can be handled separately. The stated parts are advantageously produced cost-effectively from a suitable plastics material by injection molding. Depending on the number of parts that are required, production by three-dimensional printing or rapid prototyping is also possible. Depending on the nature of the materials used, machining procedures such as milling are also applied. Individual parts or regions can also be processed by forming operations such as hot embossing. According to a further aspect of the invention, commercially available Luer parts, in particular with insertion of a sealing ring, are used as far as possible. Suitable material are, for example, biocompatible plastics. The materials that are used advantageously have a low coefficient of thermal expansion. The tube body is advantageously formed from the same material as a capillary, in particular a glass capillary. However, the tube body can also be formed from metal. Depending on the design, a conventional syringe cannula can be used as tube body.

The invention optionally also relates to a method for preparing sample material using an above-described device, in particular in a microfluidic system.

By means of biopsy needles, cells from tissues or fluids (blood, lymphatic fluid) can be removed selectively from the patient. These are then lyzed in a small volume.

Particularly in liquid biopsy applications, only a small number of cells may adhere to the needle through antibody selection, and a small volume is of great importance for sensitive analysis. If cells are to be transferred from a biopsy needle onto a microfluidic platform, the cells adhering to the needle should be lyzed in as small a volume as possible. This can take place in a glass cannula, for example. Defined volumes of lysis buffer can be drawn up. The challenge is then often the complete emptying of these cannulas, particularly if the search is for rare cells (for example tumor cells with specific point mutation, immune cells with special antigens, stem cells). The reason is that every small residue can signify a total loss of the information.

The core of the method or of the device is the loss-free processing of a limited sample volume in a cannula. The limited sample volume is drawn in by means of the rotary device and expelled again. Possible residues of fluid are displaced completely from the cannula by an oil phase via a side channel, and the entire sample volume can be further processed without any loss. The oil phase can additionally be used for the further processing on a lab-on-a-chip.

Through the use of the device described here and the processes employed, the following advantages are afforded:
a) A sample volume can be removed completely from a cannula. This is particularly advantageous when working with small volumes, when little sample material is present (for example small copy cells on DNA strands with rare mutation patterns) or when the volume for quantifying has to be exact.
b) The oil phase used can be further utilized for subsequent processes. This is an advantage particularly for further processing on a lab-on-a-chip platform that uses two-phase systems.
c) By the follow-on movement of oil through the cannula when transferring the sample to a lab-on-a-chip, the sample can be directly enclosed as a plug in several oil phases and can be further processed directly, and without loss, on the lab-on-a-chip platform.
d) Volume retention can be guaranteed by pre-storage on a lab-on-a-chip platform. The lysis unit does not have to guarantee an exact take-up. The volume retention is guaranteed solely by the volumes of the lab-on-a-chip platform.
e) The invention can be realized using commercially available, standardized Luer parts. In particular, these parts are produced from biocompatible and PCR-compatible materials. PCR denotes polymerase chain reaction.
f) The device also makes it possible to perform a multi-step lysis method without any losses.
g) The fluid method permitted by the device reduces to a minimum the manual steps that take place before the automatic on-chip processing. Nor do these manual steps have to be metrically precise. The precision is guaranteed by the volume displacement.
h) The complete and loss-free emptying avoids a readjustment of volumes and any dilutions. Readjustment always means that a volume measurement and a feedback system have to be implemented. This leads to more complex systems and to higher development costs, which it is desirable to limit in point-of-care applications.

Biocompatible plastics are suitable materials. It is obvious to use the same material from which the cartridge or a container of the microfluidic system is made. The material should have a low coefficient of thermal expansion. As has already been mentioned, a glass capillary or also a syringe cannula made of metal can be used for the capillary. Standard Luer parts can also be used. Syringes can be produced from plastic or glass but should be connected to the T-element in a sealed manner (by Luer attachment or also adhesive bonding).

Further advantages, features and details of the invention will become clear from the following description in which various illustrative embodiments are described in detail with reference to the drawing.

### BRIEF DESCRIPTION OF THE DRAWING

FIG. 1 shows a schematic view, in longitudinal section, of a device for preparing sample material, with a rotary body arranged rotatably in a hollow body;

FIG. 2 shows a schematic view, in longitudinal section, of a tube body which, with the aid of a sealing device, is connected in a fluid-tight manner to a coupling body;

FIG. 3 shows a schematic view, in longitudinal section, of a tube body which is combined with an attachment body;

FIG. 4 shows, in longitudinal section, a similar device to that of FIG. 1, with a sealing device and with two abutments;

FIG. 5 shows, in longitudinal section, a similar device to that of FIG. 4, with a sample removal device;

FIG. 6 shows a similar device to that of FIG. 5, with a symbolically indicated PCR bead on the sample removal device;

FIG. 7 shows, in longitudinal section, a similar device to that of FIG. 5, with an additional third abutment;

FIG. 8 shows, in longitudinal section, the device from FIG. 7 after the additional abutment has been passed;

FIG. 9 shows a flow diagram illustrating a method for preparing sample material using a device as shown for example in FIGS. 1 and 4 to 8;

FIG. 10 shows, in section, a microfluidic system with a lab-on-a-chip and with an open end of a tube body of the device from FIG. 1;

FIG. 11 shows, in longitudinal section, the open end of the tube body of the device from FIG. 1 with sample material, with a microfluidic container and with a magnet device;

FIG. 12 shows, in longitudinal section, the device from FIG. 5 with an additional filter device at the open end of the tube body;

FIG. 13 shows, in longitudinal section, a similar device to that of FIG. 1, with a sample removal device and with a sliding seal;

FIG. 14 shows a further illustrative embodiment of a device for preparing sample material, with an additional T-piece;

FIGS. 15 to 17 show a schematic view of a method in which the device from FIG. 14 is used to transport fluid completely out of a cannula;

FIG. 18 shows a variant of the device from FIG. 14, when a syringe is attached to the T-piece;

FIG. 19 shows a similar view to that of FIG. 18, when a syringe is attached to the T-piece from above;

FIGS. 20 to 23 show a schematic illustration of a method by which the device from FIG. 14 can advantageously be integrated, in a fluidic outlet for a lysis process with cells adhering to a biopsy needle, into a lab-on-a-chip platform; and

FIGS. 24 to 26 show a use of the device from FIG. 18, wherein two phases are stored in the syringe.

### DESCRIPTION OF THE ILLUSTRATIVE EMBODIMENTS

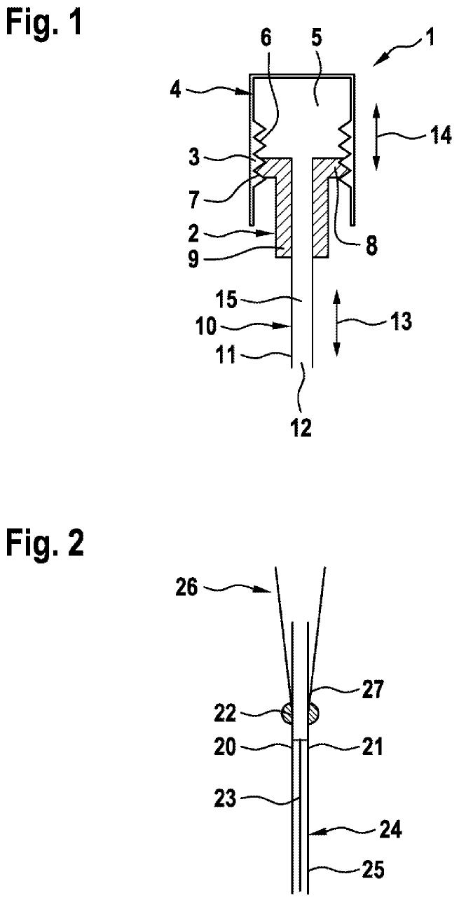

FIG. 1 shows schematically, in longitudinal section, a device 1 configured as a rotary device for preparing sample material 23, 33 (in FIGS. 2, 3, 5 to 8, 12, 13).

The rotary device 1 comprises a rotary body 2 which is rotatable in a hollow body 4 with the aid of a thread 3. The hollow body 4 has the shape of a straight circular cylinder, which is closed at its upper end in FIG. 1. The hollow body 4 delimits a liquid-receiving space 5.

The thread 3 comprises an inner thread portion 6 in the hollow body 4. An outer thread portion 7 of the thread 3 engages in the inner thread portion 6. The outer thread portion 7 is formed on a collar 8 of the rotary body 2. The collar 8 is angled away from a main body 9 of the rotary body 2.

The main body 9 of the rotary body 2 comprises a central through-hole, which leads into a tube body 10. The tube body 10 is configured, for example, as a capillary 11 with an open end 12 at the bottom. The capillary 11 delimits on the inside a sample-receiving space 15, which is fluidically connected to the liquid-receiving space 5 in the hollow body 4 via the central through-hole in the rotary body 2.

A double arrow 13 in FIG. 1 indicates that the tube body 10 moves up and down with the rotary body 2 when the rotary body 2 is rotated relative to the hollow body 4. The movement of the rotary body 2, indicated by the double arrow 13, is also designated as a stroke. The stroke of the rotary body 2 changes the volume of the liquid-receiving space 5, as is indicated by a double arrow 14 in FIG. 1.

When the volume of the liquid-receiving space 5 becomes smaller, liquid is expelled from the liquid-receiving space 5 through the sample-receiving space 15 and the open end 12 of the tube body 10. When the volume of the liquid-receiving space 5 becomes greater, liquid is drawn into the liquid-receiving space 5 through the open end 12 of the tube body 10. The liquid is made available by way of a suitable container (not shown in FIG. 1) at the open end 12 of the tube body 10.

FIG. 2 shows how a tube body 20, which is configured as a capillary 21, can be attached to a rotary body (2 in FIG. 1). To provide a fluid-tight connection, a seal 22 is mounted on the capillary 21. Above the seal 22, an end portion of the capillary 21 is arranged inside a coupling body 26. The coupling body 26 comprises a tip 27 which is configured similarly to the tip of a pipet. The free end of the tip 27 of the coupling body 26 bears sealingly on the seal 22.

The coupling body 26 is connected, for example, to a rotary body, as shown in FIG. 1 and labeled by 2. The connection between the rotary body and the coupling body 26 can be an integral connection. Sample material 23 on a sample removal device 24 is arranged in the capillary 21. The sample removal device 24 is configured as a biopsy needle 25. In FIG. 2, only a functional or functionalized portion of the biopsy needle 25 is arranged in the capillary 21.

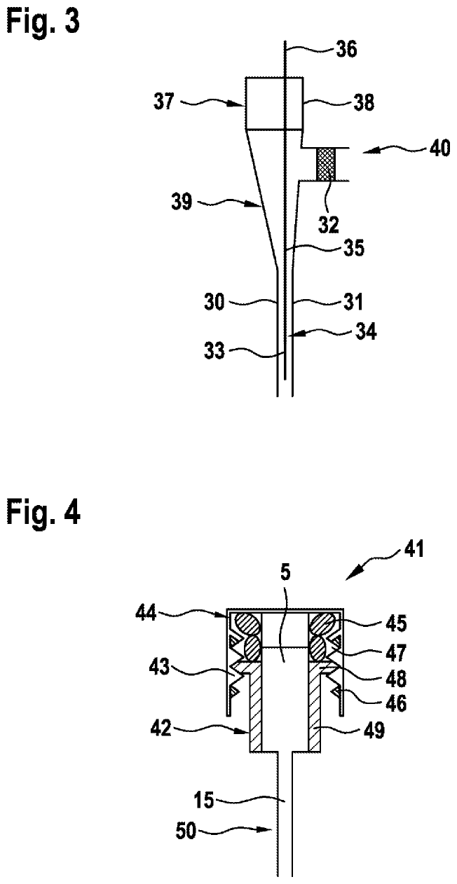

FIG. 3 shows a tube body 30 configured as a cannula 31. Sample material 33 on a sample removal device 34 is arranged in the cannula 31. The sample removal device 34 comprises, for example, a biopsy needle 35, of which the functional portion is arranged inside the cannula 31. The rest of the biopsy needle 35 is guided through an attachment body 39 with a coupling device 37.

The attachment body 39 is connected integrally to the tube body 30. On its right-hand side in FIG. 3, the attachment body 39 has an attachment 40, for example for a rotary device (not shown in FIG. 3). The attachment 40 is combined with a filter device 32.

The coupling device 37 comprises a Luer lock element 38, which comprises a push-through region for the biopsy needle 35. A free end 36 protrudes upward in FIG. 3 out of the coupling device 37.

The attachment body 39 tapers to a point toward the tube body 30. The attachment body 39, with the tube body 30 and the coupling device 37 and the filter device 32, is configured for example as a disposable part. The rotary device that is attached to the attachment 40 is then configured, for example, as a reusable part. The filter 32 in this case advantageously serves to reduce a danger of contamination.

FIG. 4 shows how a predefined volume can be drawn in with a rotary device 41. For this purpose, the rotary device 41 has to be airtight and, during the intake of liquid, must be rotated by a precisely defined number of revolutions. The rotary device 41 comprises a rotary body 42, which is coupled to a hollow body 44 via a thread 43. A sealing device 45 is arranged for sealing between the rotary body 42 and the hollow body 44.

The thread 43 is provided in the hollow body 44 with two abutments 46, 47, by which the rotation of the rotary body 42 in the hollow body 44 is limited. The rotary body 42 comprises a main body 49 with a central through-hole. In FIG. 4, a collar 48 is angled away from the top of the main body 49. At its lower end in FIG. 4, the rotary body 42 is connected to a tube body 50.

The tube body 50 comprises a sample-receiving space 15, which is connected to a liquid-receiving space 5 inside the rotary body 42 or inside the hollow body 44. As a result of the increasing space inside the rotary device 41, a negative pressure is obtained, which ensures that a precisely defined volume of liquid is sucked through the open end of the tube body 50 into the rotary device 41.

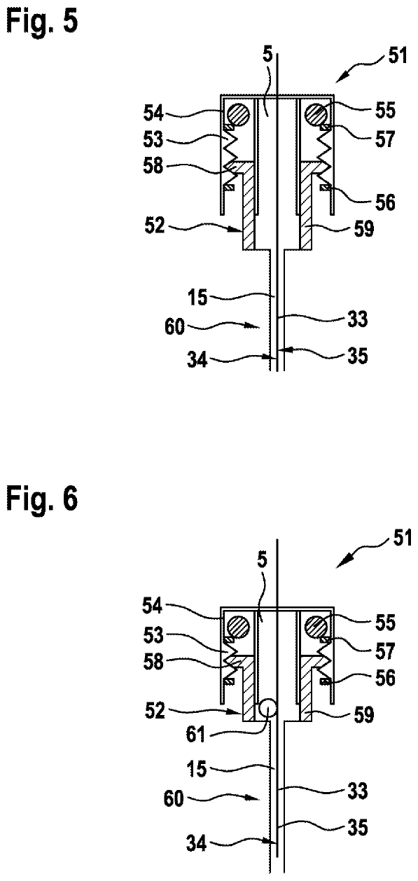

FIGS. 5 and 6 show a device 51 configured as a rotary device and having a rotary body 52 which is rotatable in a hollow body 54 via a thread 53. A sealing device 55 serves for sealing between the rotary body 52 and the hollow body 54 with the thread 53. The rotary body 52 comprises a collar 58, which is angled away from a main body 59. The main body 59 is connected to a tube body 60, which is configured as a capillary or cannula.

A sample removal device 34, which is configured as a biopsy needle 35, is arranged in the tube body 60. The sample material 33 is arranged on the sample removal device 34 and is prepared for analysis with the aid of liquid. The liquid is sucked into the liquid-receiving space 5 through the sample-receiving space 15 inside the tube body 50. For this purpose, the rotary body 52 is rotated in a defined manner. The rotation of the rotary body 52 relative to the hollow body 54 is limited by two abutments 56, 57 on the thread 53.

The rotary body 52 can also be designated as an adapter piece and is configured in FIG. 5 as a Luer lock with a rubber septum. This affords the advantage that a rigid probe, for example the biopsy needle 35, or a functionalized wire is already equipped with a Luer lock. Laborious working of the wire is thus avoided, which greatly minimizes the danger of undesired contamination or destruction of the probe. Moreover, the probe does not first of all have to be laboriously processed and can instead be used and treated directly.

FIG. 6 shows the possibility of a combination of several steps for subsequently carrying out a quantitative real-time polymerase chain reaction (PCR) within the device 51. A PCR bead 61 is, for example, provided inside the rotary body 52. Chemicals required for the preparation, for example lyophilizate, can be stored for example in dry form.

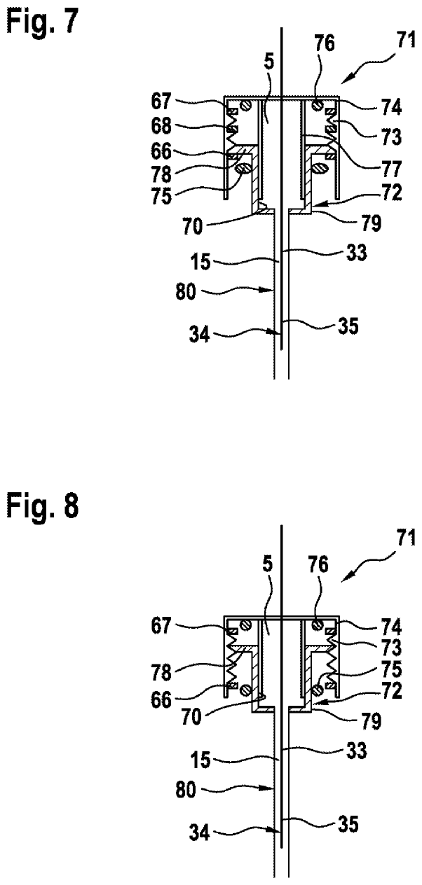

FIGS. 7 and 8 show a device 71 which is configured as a rotary device and with which a multi-step method can be carried out. The device 71 comprises a rotary body 72 which is rotatable in a hollow body 74 with the aid of a thread 73. Two sealing devices 75, 76 are provided for sealing between the hollow body 74 and the rotary body 72.

The rotary body 72 comprises a collar 78, which is angled away from a main body 79 of the rotary body 72. The main body 79 of the rotary body 72 merges into a tube body 80 which, as in the preceding illustrative embodiments, comprises the sample removal device 34.

The hollow body 74 is combined with a sealing cylinder 77. The sealing cylinder 77 has the form of a straight circular cylinder and is closed at its upper end in FIG. 7. A non-functional portion of the sample removal device extends through the closed end of the sealing cylinder 77.

The rotary body 72 comprises a central recess 70 in which the lower end of the sealing cylinder 77 in FIG. 7 engages. The sealing cylinder 77 is rigidly connected to the hollow body 74. The rotary body 72 is rotatable relative to the sealing cylinder 77. The thread 73 and the sealing devices 75, 76 are arranged in an annular space which is delimited radially to the inside by the sealing cylinder 77 and radially to the outside by the hollow body 74. The collar 78 of the rotary body 72 is rotatable via the thread 73 between a total of three abutments 66, 67, 68.

A lower abutment is designated by 66. An upper abutment is designated by 67. An additional central abutment 68 is arranged between the two abutments 66 and 67. In FIG. 7, the collar 78 of the rotary body 72 is arranged between the two abutments 66 and 68. With the different abutments 66 to 68, it is possible in a simple way to define different volumes which are expelled or drawn in by the device 71 during the rotation of the rotary body 72 relative to the hollow body 74.

When a multi-step method is carried out with the device 71 in FIG. 7, a first volume V1 is taken up once or several times for example in a first step. For this purpose, the rotary device 71 with the collar 78 is rotated from the lower abutment 66 as far as the additional central abutment 68. In a further step, a second volume V2 is metered by means of the device being rotated as far as the upper abutment 67. The second volume is greater than the first volume.

FIG. 8 shows how the rotary body 72 with the collar 78 is moved between the two abutments 66 and 67 in order to draw in or expel the second volume. The additional central abutment (68 in FIG. 7) is destroyed in FIG. 8 after a first pass and is therefore no longer present.

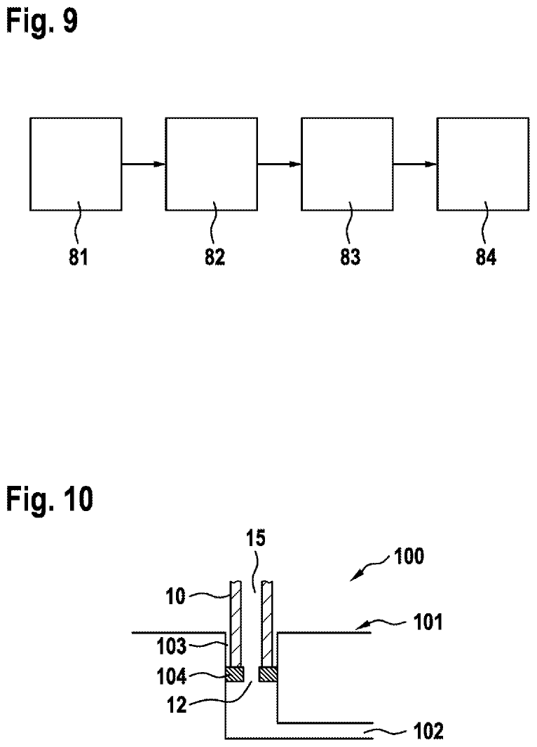

In FIG. 9, rectangles 81 to 84 illustrate a method in which the device 1; 41; 51; 71; 131 is used and by which a sample removal device 24; 34 equipped for example with cells, in particular a wire equipped with cells, is prepared for a genetic analysis. Here, the cells are intended to be lyzed in the device, and the lysate is to be transferred into a microfluidic analysis unit. The rectangle 81 indicates a washing step. The probe needle is washed, for example, with a phosphate-buffered saline solution in order to remove any possible residues from the sample, for example blood, fat, culture medium. For this purpose, the washing liquid is drawn into the device through the open end of the tube body and expelled again.

The rectangle 82 indicates a fixing step. In the fixing step, the sample is biologically fixed such that no further biochemical reactions take place in the cells.

For this purpose, a fixing solution, for example formaldehyde or acetone, is taken up by the device, incubated and expelled again. This is advantageously followed by a further brief washing step.

The rectangle 83 indicates a lysis step. In the lysis step, internal cell material, such as proteins or nucleic acids, is released by the lysis. For this purpose, a lysis solution, for example distilled water, is taken up and the cells incubated therein.

The rectangle 84 indicates a sample transfer step. Here, the lysate from step 83 is transferred directly into a microfluidic analysis unit. The microfluidic analysis unit belongs to a microfluidic system, as is designated by 100 in FIG. 10.

The microfluidic system 100 in FIG. 10 comprises a lab-on-a-chip 101. The lab-on-a-chip 101 comprises a microfluidic channel system 102. For the sample transfer, the tube body 10 is arranged with its opening 12 in an insertion opening 103 of the lab-on-a-chip 101. The positioning of the tube body 10 is made easier by an abutment 104 in the insertion opening 103.

With the aid of the rotary device, a defined quantity of fluid, in particular liquid, for preparing a sample can then be easily drawn in from the lab-on-a-chip 101. After the sample has been prepared, it can then be expelled, likewise with the aid of the rotary device, into a corresponding sample chamber of the lab-on-a-chip 101.

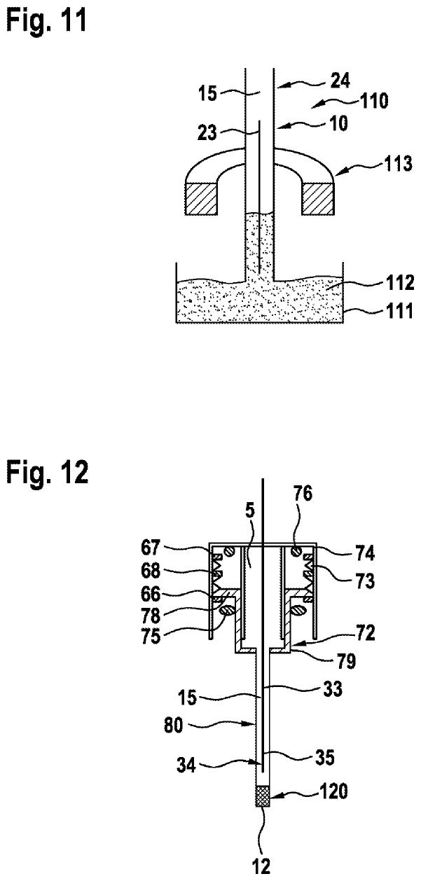

FIG. 11 shows a use in a microfluidic system 110 with possible magnetic cleaning. The microfluidic system 110 comprises a microfluidic container 111 with magnetic beads 112. In the magnetic cleaning, the magnetic beads 112 are introduced with a functionalized surface into the tube body 10 and expelled again. Depending on the nature of the functionalization, the beads bind either disruptive constituent parts or the desired sample. For this purpose, the tube body 10, configured for example as a cannula, is advantageously enclosed by a magnetic device 113. The magnetic beads 112 are transported in the tube body 10 by a corresponding movement of the magnetic device 113.

Using the example of the device 71 illustrated in FIG. 7, FIG. 12 shows that a filter device 120 can also be placed at the open end 12 of the tube body 80. Purification or pre-purification of the sample can be easily carried out with the filter device 120.

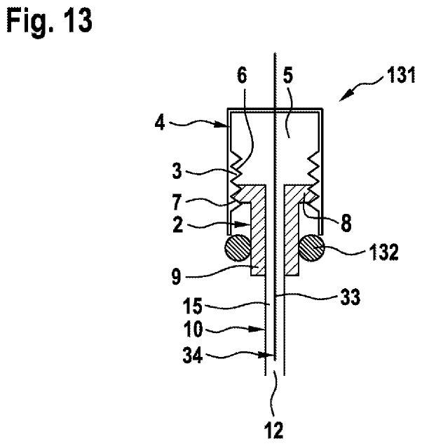

FIG. 13 shows a device 131 similar to the device 1 from FIG. 1. The same reference signs as in FIG. 1 are used to designate the same or similar parts. To avoid repetition, reference is made to the above description of FIG. 1.

A sample removal device 34 with sample material 33 is arranged in the device 131. In contrast to the device 1 in FIG. 1, the device 131 comprises a sliding seal 132 for the sealing between the rotary body 2 and the hollow body 4. The sliding seal 132 permits relatively simple sealing and is advantageously secured on the hollow body 4.

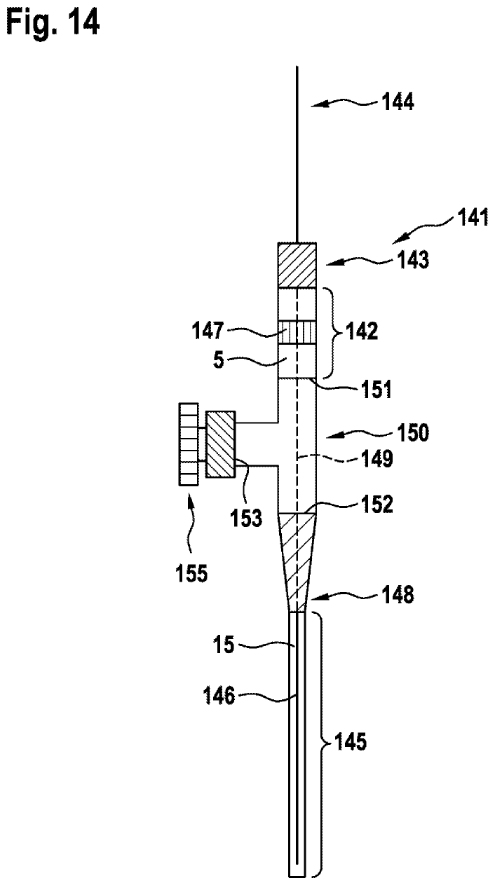

In FIGS. 14 to 26, a device 141 for preparing sample material with a rotary device 142 is shown schematically in different configurations and uses. At its upper end in the figures, the device 141 is closed in a fluid-tight and pressure-tight manner by a septum 143. The septum 143 is, for example, a stopper made of an elastic material through which a biopsy needle 144 is pushed.

The biopsy needle 144 extends through the septum 143 into the interior of the device 141. A portion 146 of the biopsy needle 144 is arranged in a functional region 145 of the device 141. The portion 146 of the biopsy needle 144 is preferably a functionalized portion.

The rotary device 142 comprises a rotary body 147 which, as has been described above, is movable in an axial direction, i.e. downward and upward in FIGS. 14 to 26, via a thread (not shown in FIGS. 14 to 26), when the rotary body 147 is rotated.

The functional region 145 of the device 141 is configured as a sample-receiving body 148. The sample-receiving body 148 can also be designated as a tube body and is configured, for example, as a capillary.

Between the sample-receiving body 148 and the rotary device 142, the device 141 comprises a T-piece 150. The T-piece 150 has a first attachment 151 for the sample-receiving body 148, and a second attachment 152 for the rotary device 142. As is indicated in FIG. 14 by a broken line, the biopsy needle 144 extends lengthwise from the top downward through the septum 143, through the rotary body 147 and through the T-piece 150 into the functional region 145 of the device 141.

The T-piece 150 comprises a third attachment 153 which, in FIG. 14, is closed in a fluid-tight and pressure-tight manner by a closure body 155. In FIGS. 14 to 18, 21, 22 and 24 to 26, the third attachment 153 of the T-piece 150 is arranged perpendicularly or transversely with respect to the longitudinal extent of the device 141.

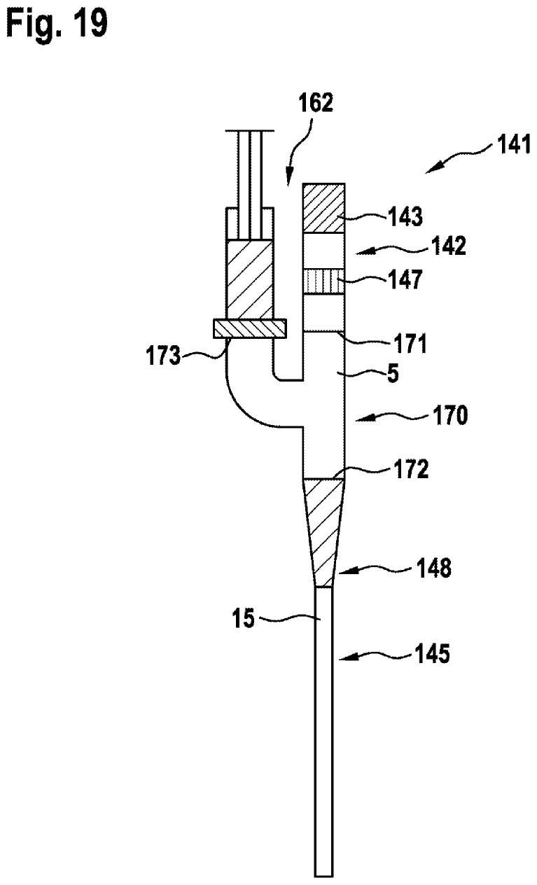

FIG. 19 shows the device 141 with a T-piece 170 in which attachments 171 and 172 correspond to the attachments 151 and 152 of the T-piece 150. In contrast to the T-piece 150, a third attachment 173 of the T-piece 170 is arranged parallel to the longitudinal extent or longitudinal axis of the device 141.

The sample-receiving body 148 in FIG. 14 is, for example, a cannula in which the functionalized biopsy needle 144 with sample material is arranged for fluidic processing. For this purpose, the sample-receiving body 148 is attached via the T-piece 150 to the rotary device 142 with which, by a rotational movement of the rotary body 147, fluid can be drawn in through the open end of the cannula 148 and expelled.

The third attachment 153 of the T-piece 150 makes available a channel which, during the operation of the device 141, can be used to convey liquids from above through the T-piece 150 into the cannula 148. In this way, a flow or stream through the cannula 148 can be easily generated.

The closure body 155 is configured, for example, as a rotary closure cap and is preferably standardized for Luer parts. If so required, the closure body 155 can be unscrewed in order to introduce a fluid, with a suitable device such as a syringe, into the device 141 through the third attachment 153 of the T-piece 150.

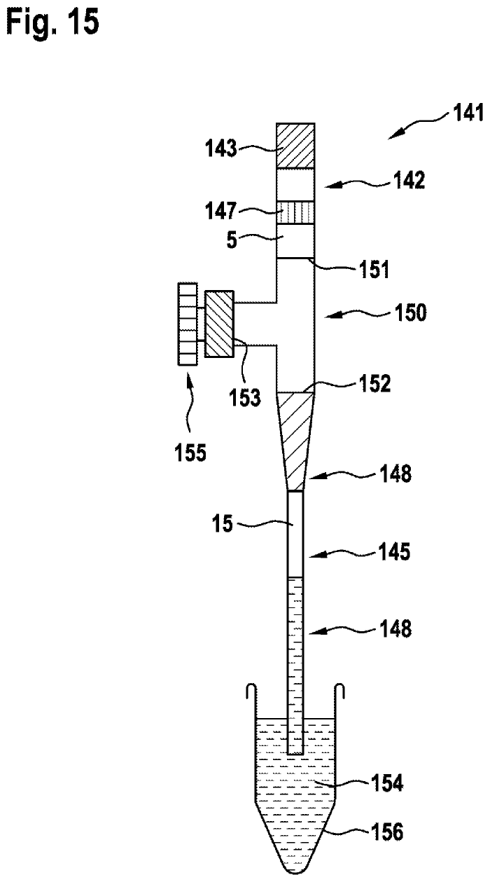

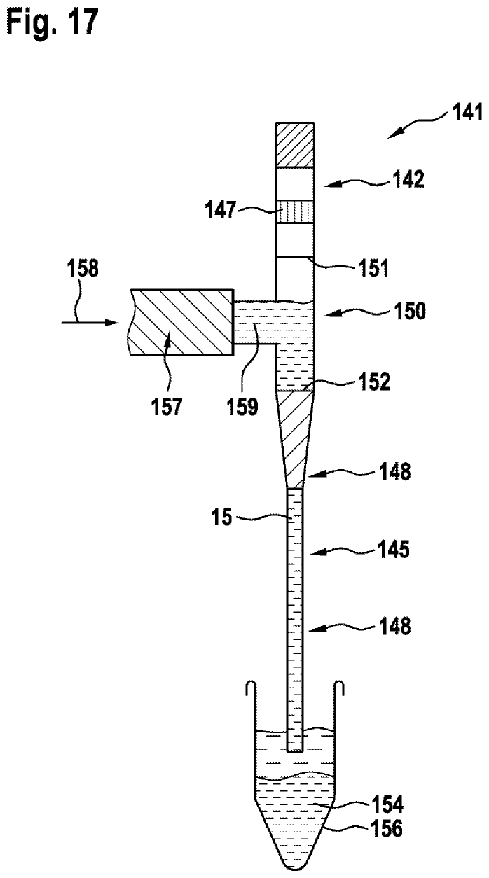

FIGS. 15 to 17 show how the device 141 from FIG. 14 is used to completely remove a fluid, in particular a liquid 154 with the sample, from the cannula 148. For this purpose, as is indicated in FIG. 17, an inert phase 159, in particular an oil phase, is transported through the cannula 148 in order to collect the fluid, in particular the sample material 154, in a container 156 at the open end of the cannula 148. The container 156 preferably belongs to a lab-on-a-chip.

The method shown in FIGS. 15 to 17 is particularly of importance in connection with a biopsy needle when the material adhering to the biopsy needle, generally cells, is to be transferred in the smallest possible volume and the entire volume is to be further processed. Lysis in a small volume is important particularly in the enrichment of rare cells, for example circulating tumor cells with defined mutations, immune cells with defined epitopes and antigens or stem cells.

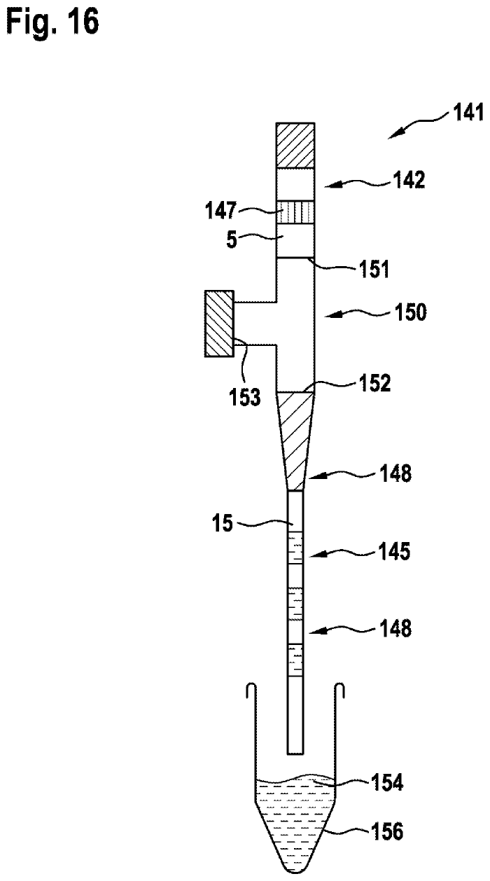

In addition, the lysate should be able to be further processed without losses. With the device 141, fluid can be taken up into the cannula 148 with the aid of the rotary device 142, as is seen in FIG. 15. Possible lysate residues, for example individual droplets, may remain in the cannula 148, as is indicated in FIG. 16. As can be seen in FIG. 17, these lysate residues are displaced from the cannula 148 by the follow-on movement of oil, until the entire lysate is collected in the container.

The follow-on movement of the oil phase takes place via a fluid delivery device 157, as is indicated in FIG. 17 by an arrow 158. A slow follow-on movement of the oil phase prevents undesired mixing in the two-phase system. Thus, the oil can then be decanted off by a phase separation or can be further used as a seal.

If a defined quantity of lysis buffer is stored in the container 156, it is then also possible for only a proportion of this volume to be drawn in for lysis and, as described above, returned completely into the storage vessel or the container 156. Volume retention is thus permitted, as a result of which it is possible to dispense with complicated volume adjustment and complicated volume measurements.

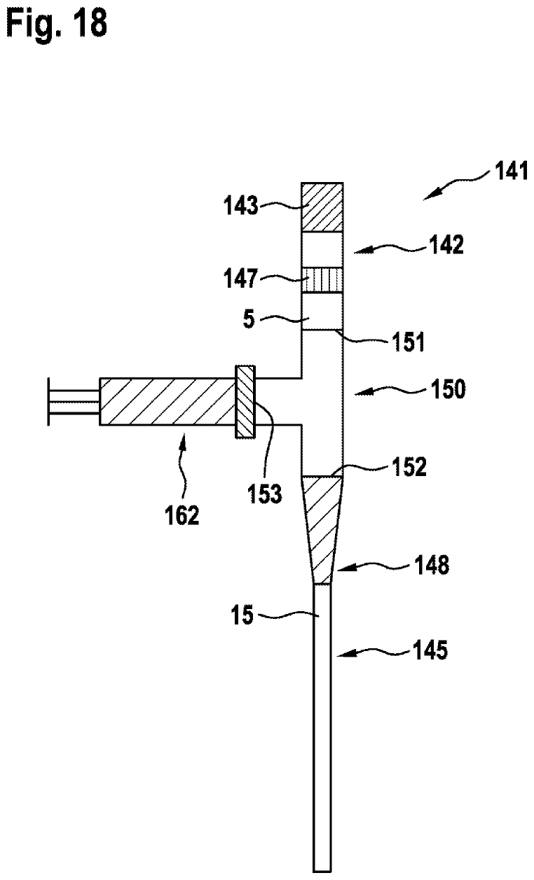

FIGS. 18 and 19 show that the oil phase can be introduced into the device 141 via the attachment 153; 173 with the aid of a syringe 162. The syringe 162 is advantageously configured as a Luer part and can be screwed instead of the closure body (155 in FIG. 14) onto the third attachment 153; 173 of the T-piece 150; 170. Thus, the syringe 162 can be used as a fluid delivery device. In order to apply the oil phase, a piston of the syringe 162 can be actuated. In FIG. 18, the syringe 162 is arranged orthogonally with respect to the biopsy needle. In FIG. 19, the syringe 162 is arranged parallel to the biopsy needle on account of the different configuration of the T-piece 170.

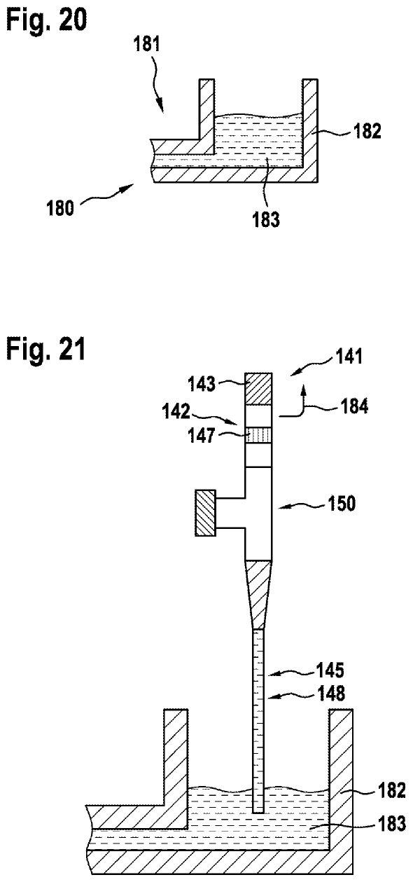

FIGS. 20 to 23 show a method by which the device 141, advantageously in a fluidic outlet for a lysis process with cells adhering to a biopsy needle, can be integrated into a lab-on-a-chip platform. In the schematically indicated process, a liquid phase 183 is stored in a sample input chamber 182. The sample input chamber 182 is provided, for example, in a lab-on-a-chip 181 of a microfluidic system 180.

The liquid phase or liquid 183 is a lysis buffer which is present in the volume, in order later to provide a lyophilized bead with the chemicals for a subsequent analysis reaction, for example sequencing. The above-described device 141 with the biopsy needle (not shown in FIGS. 20 to 23) is then used to draw in as much lysis buffer 183 as is needed to fill the cannula completely, as can be seen in FIG. 21. The lysis can also be performed by repeated raising and lowering of lysis buffer. The stroke in the raising and lowering of the lysis is initiated by rotation of the rotary body 147, as is indicated in FIG. 21 by an arrow 184.

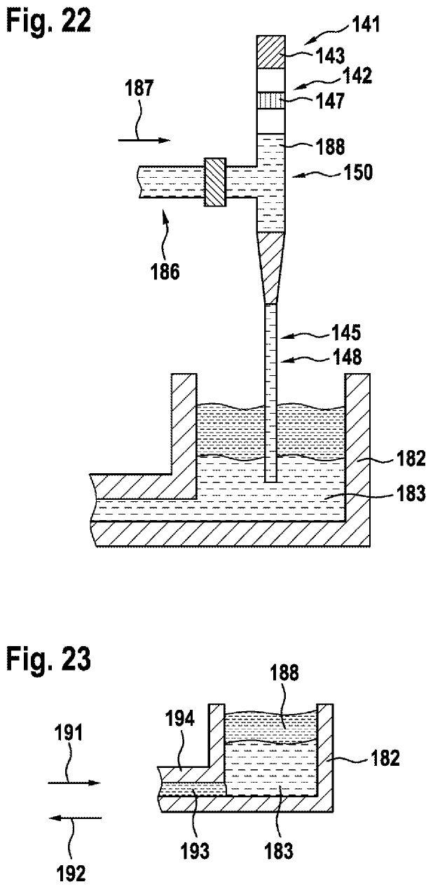

Then, as is indicated in FIG. 22 by an arrow 187, the lysate is expelled completely from the device 141 by oil 188. As a result of phase separation, the now preserved lysis buffer volume, including cell material, is located in the sample input chamber 182, which is also designated as storage chamber. The lysis buffer volume is then additionally overlaid by the oil.

As is indicated in FIG. 23 by arrows 191 and 192, the sample input chamber 182 be controlled by suitable microfluidics of the microfluidic system 180. Here, a feed channel 194 of the sample input chamber 182 is filled by means of oil 193. The lysate is then enclosed between two oil phases and can be transported, without loss, in the lab-on-a-chip system 181.

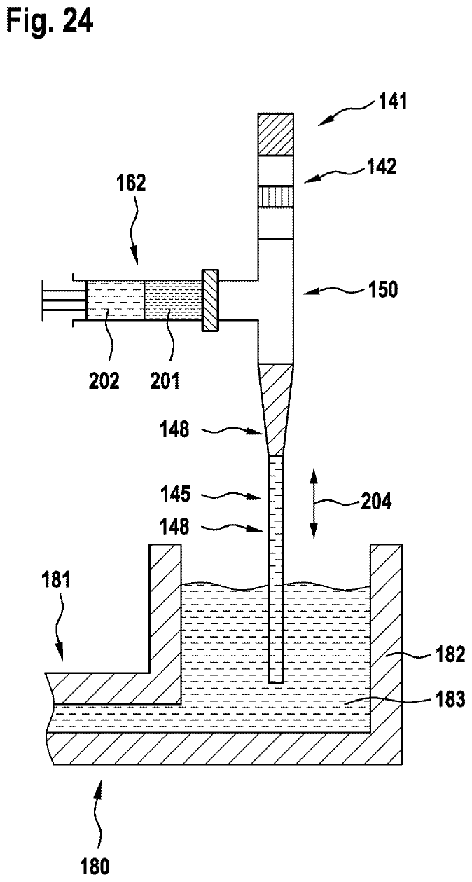

FIGS. 24 to 26 show the use with a syringe 162 in which a first phase 201 and a second phase 202 are stored. The first phase 201 is an aqueous phase, for example. The second phase 202 is an oil phase, for example. A phase separation is advantageously achieved by a parallel arrangement of the syringe 162, as is shown in FIG. 19. The desired phase separation has the effect that the two phases 201, 202 do not mix in the syringe 162.

A double arrow 204 in FIG. 24 indicates that the device 141 can be used with the rotary device 142 in order to draw fluid in and to expel fluid.

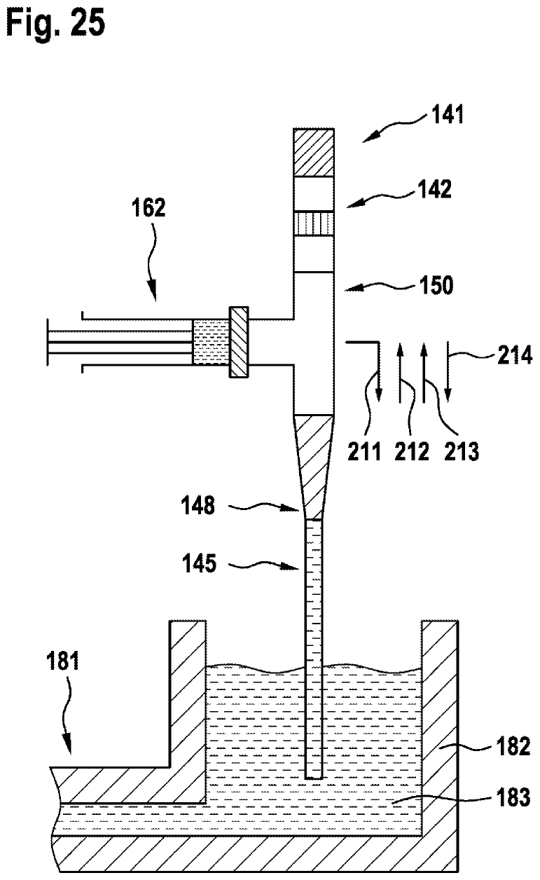

An arrow 211 in FIG. 25 indicates that the for example aqueous phase 201 is first of all pushed through the cannula 148 and can be pumped up and down with the aid of the syringe 162, as is indicated by arrows 212 to 214 in FIG. 25. The lysis buffer and the lysate thus mix with the liquid 201. This is particularly of interest when using a lysis method that consists of several steps. Thus, for example, basic lysis buffers are neutralized by an acid buffer before the lysate is further processed.

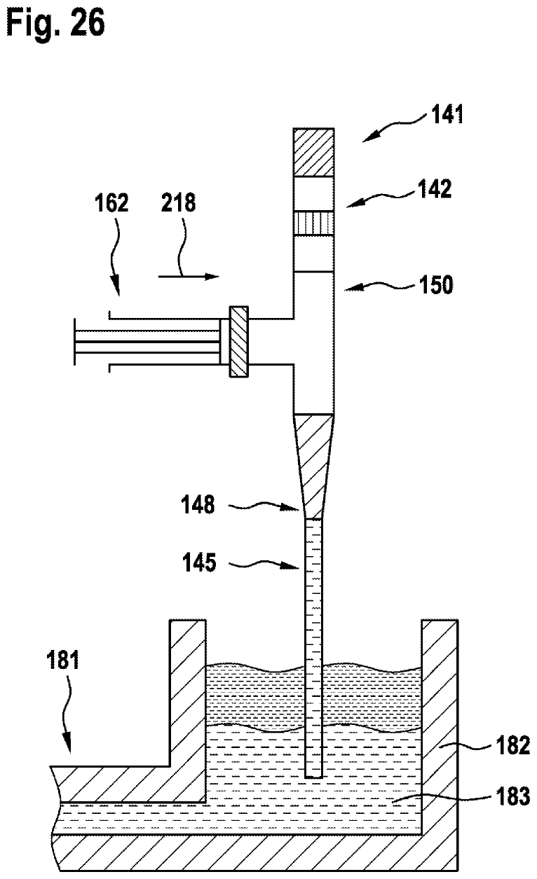

An arrow 218 in FIG. 26 indicates that, at the end of the method, the second phase or oil phase 202 is introduced from the syringe 162 into the device 141.

1. A rotary device for preparing sample material comprising:
a sample-receiving space for the sample material,
wherein the rotary device is configured to perform a rotational movement to draw in a defined quantity of liquid into the sample-receiving space or expel the defined quantity of liquid from the sample-receiving space.

2. The rotary device as claimed in claim 1, further comprising:
a liquid-receiving space; and
a rotary body coupled to the liquid-receiving space by a thread, the liquid-receiving space having a volume with a size that is altered by rotation of the rotary body relative to the thread or by rotation of the thread relative to the rotary body.

3. The rotary device as claimed in claim 2, further comprising:
a hollow body defining the liquid-receiving space and that is equipped internally with the thread.

4. The rotary device as claimed in claim 3, wherein the hollow body has a sealed push-through region at an end directed away from the sample-receiving space.

5. The rotary device as claimed in claim 4, wherein the sealed push-through region has a needle carrying the sample material.

6. The rotary device as claimed in claim 2, wherein the liquid-receiving space is fluidically connected to the sample-receiving space.

7. The rotary device as claimed in claim 2, further comprising:
a tube body defining the sample-receiving space, the tube body being open at its an directed away from the liquid-receiving space.

8. The rotary device as claimed in claim 7, wherein one of the tube body and the rotary body has an outer thread portion which complements an inner thread portion arranged in the liquid-receiving space.

9. The rotary device as claimed in claim 7, wherein at least one of the tube body, a hollow body that defines the liquid-receiving space, and the rotary body is combined with at least one sealing device.

10. The rotary device as claimed in claim 7, wherein at least one of the tube body, a hollow body that defines the liquid-receiving space, and the rotary body is combined with a filter device.

11. The rotary device as claimed in claim 2, wherein one of the liquid-receiving space and the sample-receiving space has an attachment configured for delivery and/or discharge of a fluid.

12. The rotary device as claimed in claim 11, wherein the attachment is configured as a third attachment on a T-piece, which delimits the liquid-receiving space and/or the sample-receiving space.

13. The rotary device as claimed in claim 12, wherein the T-piece has a first attachment for the rotary device, a second attachment for the sample-receiving space, and the third attachment for delivery and/or discharge of the fluid.

14. A microfluidic system comprising:
a rotary device for preparing sample material, the device comprising: a sample-receiving space for the sample material,
wherein the rotary device is configured to perform a rotational movement to draw in a defined quantity of liquid into the sample-receiving space or expel the defined quantity of liquid from the sample-receiving space.

15. A method for preparing sample material comprising:
using a rotary device, which has a sample-receiving space for receiving the sample material, to perform a rotational movement to draw in a defined quantity of liquid into the sample receiving space or expel the defined quantity of liquid from the sample receiving space.
